(19) United States
(12) Patent Application Publication
(10) Pub. No.: US 2026/0201458 A1
(43) Pub. Date: Jul. 16, 2026

(54) METAGENOMIC FILTERING FOR MONITORING AND MODULATING ENVIRONMENTAL MICROBIOMES

(71) Applicant: MARS, INCORPORATED, McLean, VA (US)

(72) Inventor: Balasubramanian GANESAN, Vancouver, WA (US)

(73) Assignee: MARS, INCORPORATED, McLean, VA (US)

(21) Appl. No.: 19/133,646

(22) PCT Filed: Nov. 27, 2023
(86) PCT No.: PCT/US2023/081109

§ 371 Date: May 28, 2025

Related U.S. Application Data
(60) Provisional application No. 63/429,393, filed on Dec. 1, 2022.

(51) Int. Cl. C12Q 1/6869; C40B 40/06; C40B 50/00; G16B 20/00; G16B 30/10

(52) U.S. Cl. CPC C12Q 1/6869; C40B 40/06; C40B 50/00; G16B 20/00; G16B 30/10

(57) ABSTRACT

Methods and systems for determining and modulating one or more microbial metabolic activities in an environment are disclosed herein. The methods disclosed herein can be applied to a wide range of environments, including, but not limited to, aquatic environments, soil environments, and factory effluents, and may be useful for biological remediation of polluted sites or management of biological oxygen demand in factory effluents. The methods comprise obtaining one or more samples from one or more locations in an environment, sequencing a plurality of nucleic acid sequences within the samples, identifying one or more microbial metabolic signatures within the plurality of nucleic acid sequences, comparing the identified microbial metabolic signatures against one or more databases, determining one or more microbial metabolic activities that correspond to one or more of the microbial metabolic signatures identified and, optionally, modulating the microbiome in the environment to increase the determined microbial metabolic activity.

### CROSS-REFERENCE TO RELATED APPLICATIONS

This application claims the benefit of U.S. Provisional Application No. 63/429,393 filed Dec. 1, 2022, the entire contents which are incorporated herein by reference.

### FIELD OF THE DISCLOSURE

This disclosure relates to methods and systems for detecting unwanted microbes and origins thereof in an environment using microbiome analysis and metagenomic data filtering.

This disclosure relates to methods and systems for determining and modulating microbial metabolic activities in environments using microbiome analysis and metagenomic data filtering.

### BACKGROUND OF THE DISCLOSURE

Microbial communities, also known as microbiomes or microbiota, are rich sources of biodiversity and perform complex metabolic and nutrient cycling processes. There is an increasing interest in understanding how microbiomes function in external environments in order to leverage the underlying metabolic processes to solve environmental issues. For example, bioremediation is the process of utilizing microbes to modulate microbiomes for the removal of pollutants from the environment and the restoration of clean environments. Bioremediation has been successfully used for removing toxic compounds from various soil and aquatic environments, including the cleanup of oil spills from ocean oil drilling platforms. Similar concepts are also utilized in closed-system waste treatment facilities, such as wastewater treatment and factory effluent treatment plants. However, these efforts are complicated by the technical difficulty of understanding microbiomes, their compositions, and the complex functional and metabolic networks that exist within them. Therefore, efficient and reliable techniques for understanding microbiomes and microbial metabolic activities are needed to drive their application towards solving environmental issues. Existing methods include chemical methods of measuring metabolic flux in complex microbiomes, which are low-throughput and may lack sensitivity to detect metabolic pathway intermediates and identify microbes associated with specific activities. Nucleic acid-based methods have also been developed for targeted detection of known enzymes involved in specific metabolic pathways via identification of environmental microbes by DNA or RNA sequencing or proteomics. However, these methods require that known microbes or enzymes associated with specific metabolic pathways are present at a certain minimum abundance to be detected, which results in some microbes and metabolic activities going undetected.

Accordingly, there is a need for applying new methods and their outcomes for bioremediation and other purposes, such as sensitive and high-throughput methods for analyzing microbial communities of unknown composition and yet to be identified microbes and their metabolic pathways to enable modulation of microbiomes and attain desirable mitigations of environmental issues.

### SUMMARY OF THE INVENTION

Disclosed herein are systems and methods for determining and modulating one or more microbial metabolic activities in an environment. The methods and systems can be used for determining and modulating microbial metabolic activities in a wide range of environments including, but not limited to, aquatic environments, soil environments, and effluents. The methods and systems disclosed herein are based on the analysis of microbial metabolic signatures corresponding to one or more microbial metabolic activities present in a sample, such as activities for degrading toxic pollutants or lowering biological oxygen demand in effluents and aquatic environments. Environments that have microbial communities contain different microbial metabolic signatures that can be detected by sequencing nucleic acids in environmental samples. By using metagenomic data analysis, these methods can be used to detect and measure particular microbial metabolic activities in environments by identifying patterns and changes in microbial communities associated with the particular microbial metabolic activity. The determination of the microbial metabolic activity can then be used to inform modulation of the microbiome in the environment to promote beneficial microbial metabolic activities, for example, for bioremediation purposes.

In one aspect, described herein is a method for determining one or more microbial metabolic activities of one or more microbes in an environment, the method comprising: obtaining one or more samples from one or more locations in the environment associated with one or more microbial metabolic activities of one or more microbes; sequencing a plurality of nucleic acid sequences within the one or more samples; identifying one or more microbial metabolic signatures within the plurality of nucleic acid sequences; comparing the one or more identified microbial metabolic signatures against one or more databases of microbial metabolic signatures that correspond particular microbial metabolic activities of microbes; and determining one or more microbial metabolic activities of one or more microbes that correspond to one or more of the identified microbial metabolic signatures based on the comparison.

In another aspect, provided herein is a method for modulating one or more microbial metabolic activities of one or more microbes in an environment, the method comprising: determining one or more microbial metabolic activities of one or more microbes in the environment by: obtaining one or more samples from one or more locations in the environment associated with one or more microbial metabolic activities of one or more microbes, sequencing a plurality of nucleic acid sequences within the one or more samples, identifying one or more microbial metabolic signatures within the plurality of nucleic acid sequences, comparing the one or more identified microbial metabolic signatures against one or more databases of microbial metabolic signatures that correspond to particular microbial metabolic activities of microbes, and determining one or more microbial metabolic activities of one or more microbes that correspond to one or more of the identified microbial metabolic signatures based on the comparison; and modulating one or more of the determined microbial metabolic activities of the one or more microbes in the environment by modulating the microbiome in the environment. In some embodiments, the method further comprises repeating the step of determining one or more microbial metabolic activities of one or more microbes in the environment at least once. In some embodiments, the method further comprises repeating the step of modulating one or more of the determined microbial metabolic activities of the one or more microbes in the environment by modulating the microbiome in the environment at least once. In some embodiments, modulating the microbiome in the environment comprises adding microbes to the environment, adding enzymes to the environment, adding metabolite compounds to the environment, adding substrates to the environment, altering the conditions of the environment, or any combination thereof to modulate one or more of the determined microbial metabolic activities. In certain embodiments, modulating the microbiome comprises adding microbes, enzymes, substrates, and/or metabolite compounds that are associated with one or more of the determined microbial metabolic activities.

In some embodiments of the foregoing methods, the one or more microbial metabolic signatures comprise nucleic acid sequences other than sequences encoding enzymes known to be associated with the one or more microbial metabolic activities. In certain embodiments, the one or more microbial metabolic signatures do not comprise a nucleic acid sequence encoding an enzyme known to be associated with one or more of the microbial metabolic activities.

In some embodiments of the foregoing methods, the one or more microbial metabolic signatures correspond to one or more microbes associated with the one or more microbial metabolic activities. In certain embodiments, the one or more microbial metabolic signatures correspond to two, three, four, five, ten, or more microbes associated with the one or more microbial metabolic activities. In certain embodiments, the one or more microbial metabolic signatures correspond to the relative abundance of the one or more microbes associated with the one or more microbial metabolic activities.

In certain embodiments of the foregoing methods, the one or more microbial metabolic activities comprise a microbial metabolic activity for removing one or more toxins from an environment. Accordingly, described herein is a method for removing one or more toxins from an environment, the method comprising: identifying the presence of the one or more toxins in the environment; determining one or more microbial metabolic activities of one or more microbes for removing the one or more toxins in the environment by: obtaining one or more samples from one or more locations in the environment associated with one or more microbial metabolic activities of one or more microbes for removing the one or more toxins, sequencing a plurality of nucleic acid sequences within the one or more samples, identifying one or more microbial metabolic signatures within the plurality of nucleic acid sequences, comparing the one or more identified microbial metabolic signatures against one or more databases of microbial metabolic signatures that correspond to particular microbial metabolic activities of microbes for removing the one or more toxins, and determining one or more microbial metabolic activities of one or more microbes for removing the one or more toxins that correspond to one or more of the identified microbial metabolic signatures based on the comparison; and removing the one or more toxins from the environment by modulating the microbiome in the environment to increase one or more of the determined microbial metabolic activities for removing the one or more toxins in the environment. In some embodiments, the method further comprises repeating the step of identifying the presence of the one or more toxins in the environment at least once. In some embodiments, the method further comprises repeating the step of determining one or more microbial metabolic activities of one or more microbes for removing the one or more toxins in the environment at least once. In some embodiments, the method further comprises repeating the step of removing the one or more toxins from the environment by modulating the microbiome in the environment to increase one or more of the determined microbial metabolic activities for removing the one or more toxins in the environment at least once. In some embodiments, the environment is a soil environment or an aquatic environment.

In some embodiments of the method for removing one or more toxins from an environment, the one or more microbial metabolic signatures comprise nucleic acid sequences other than sequences encoding enzymes known to be associated with the one or more microbial metabolic activities for removing the one or more toxins. In certain embodiments, the one or more microbial metabolic signatures do not comprise a nucleic acid sequence encoding an enzyme known to be associated with one or more of the microbial metabolic activities for removing the one or more toxins. In certain embodiments, the one or more microbial metabolic signatures correspond to one or more microbes associated with the one or more microbial metabolic activities for removing the one or more toxins. In some variations, the one or more microbial metabolic signatures correspond to two, three, four, five, ten, or more microbes associated with the one or more microbial metabolic activities for removing the one or more toxins. In certain embodiments, the one or more microbial metabolic signatures correspond to the relative abundance of the one or more microbes associated with the one or more microbial metabolic activities for removing the one or more toxins.

In some embodiments of the method for removing one or more toxins from an environment, modulating the microbiome in the environment comprises adding microbes to the environment, adding enzymes to the environment, adding metabolite compounds to the environment, adding substrates to the environment, altering the conditions of the environment, or any combination thereof to increase one or more of the determined microbial metabolic activities for removing the one or more toxins. In certain embodiments, modulating the microbiome in the environment comprises adding microbes, enzymes, substrates, and/or metabolite compounds that are associated with one or more of the determined microbial metabolic activities for removing the one or more toxins.

In some embodiments of the method for removing one or more toxins from an environment, the one or more microbial metabolic activities for removing the one or more toxins comprise degrading the one or more toxins, sequestering the one or more toxins, neutralizing the one or more toxins, metabolizing the one or more toxins to produce one or more non-toxic compounds, or any combination thereof. In certain embodiments, the one or more toxins comprise an aflatoxin, a toxic hydrocarbon, or a combination thereof.

In certain embodiments of the foregoing methods, the one or more microbial metabolic activities comprise a microbial metabolic activity for lowering biological oxygen demand (BOD) in an effluent. Accordingly, described herein is a method for lowering BOD in an effluent, the method comprising: determining one or more microbial metabolic activities of one or more microbes for lowering BOD in the effluent by: obtaining one or more samples from the effluent associated with one or more microbial metabolic activities of one or more microbes for lowering BOD, sequencing a plurality of nucleic acid sequences within the one or more samples, identifying one or more microbial metabolic signatures within the plurality of nucleic acid sequences, comparing the one or more identified microbial metabolic signatures against one or more databases of microbial metabolic signatures that correspond to particular microbial metabolic activities of microbes for lowering BOD, and determining one or more microbial metabolic activities of one or more microbes for lowering BOD that correspond to one or more of the identified microbial metabolic signatures based on the comparison; and lowering BOD in the effluent by modulating the microbiome in the effluent to increase one or more of the determined microbial metabolic activities for lowering BOD in the effluent. In some embodiments, the method further comprises repeating the step of determining one or more microbial metabolic activities of one or more microbes for lowering BOD in the effluent at least once. In some embodiments, the method further comprises repeating the step of lowering BOD in the effluent by modulating the microbiome in the effluent to increase one or more of the determined microbial metabolic activities for lowering BOD in the effluent at least once.

In some embodiments of the method for lowering BOD in an effluent, the one or more microbial metabolic signatures correspond to one or more microbes associated with the one or more microbial metabolic activities for lowering BOD. In certain embodiments, the one or more microbial metabolic signatures correspond to two, three, four, five, ten, or more microbes associated with the one or more microbial metabolic activities for lowering BOD.

In certain embodiments, the one or more microbial metabolic signatures correspond to the relative abundance of the one or more microbes associated with the one or more microbial metabolic activities for lowering BOD.

In some embodiments of the method for lowering BOD in an effluent, modulating the microbiome in the effluent comprises adding microbes to the effluent, adding enzymes to the effluent, adding metabolite compounds to the effluent, adding substrates to the effluent, altering the conditions of the effluent, or any combination thereof to increase one or more of the determined microbial metabolic activities for lowering BOD. In certain embodiments, modulating the microbiome in the effluent comprises adding microbes, enzymes, substrates, and/or metabolite compounds that are associated with one or more of the determined microbial metabolic activities for lowering BOD. In some variations, modulating the microbiome in the effluent comprises adding anoxic bacteria to the effluent.

In some embodiments of the method for lowering BOD in an effluent, the effluent is a dairy factory effluent, a meat processing factory effluent, a plant-based food processing factory effluent, or a manufacturing effluent. In certain embodiments, the one or more microbial metabolic activities for lowering BOD comprise one or more activities for degrading actin, myosin, cellulose, animal lipids, plant proteins, biochemical effluent components, lactose, milk lipids, whey proteins, caseins, bio-available solids, or any combination thereof.

In some embodiments of any of the foregoing methods, wherein sequencing the plurality of nucleic acid sequences within the one or more samples comprises preparing a sequencing library. In some embodiments of any of the foregoing methods, sequencing the plurality of nucleic acid sequences within the one or more samples comprises next generation sequencing or microarray analysis. In some embodiments of any of the foregoing methods, the plurality of nucleic acid sequences comprise DNA sequences, RNA sequences, or a combination thereof. In some embodiments of any of the foregoing methods, non-microbial sequences are filtered from the plurality of nucleic acid sequences prior to identifying the one or more microbial metabolic signatures. In some embodiments of any of the foregoing methods, the one or more databases are databases of microbial nucleic acid sequences.

In another aspect, described herein is a system for determining the one or more microbial metabolic activities in an environment, comprising: one or more processors; and a memory comprising instructions executable by the one or more processors that, when executed by the one or more processors, cause the system to: identify one or more microbial metabolic signatures within a plurality of nucleic acid sequences obtained from sequencing a plurality of nucleic acid sequences within one or more samples from one or more locations in the environment associated with one or more microbial metabolic activities of one or more microbes; compare the one or more identified microbial metabolic signatures against one or more databases of microbial metabolic signatures that correspond to particular microbial metabolic activities of microbes; and determine one or more microbial metabolic activities of one or more microbes that correspond to one or more of the identified microbial metabolic signatures based on the comparison. In certain embodiments, the one or more microbial metabolic activities comprise a microbial metabolic activity for removing one or more toxins from an environment or for lowering biological oxygen demand (BOD) in an effluent.

### BRIEF DESCRIPTION OF THE FIGURES

FIG. 1 is a flow diagram depicting a method of determining one or more microbial metabolic activities of one or more microbes in an environment.

FIG. 2 is a flow diagram depicting an exemplary data analysis process for the identification of microbial metabolic signature in a sample from an environment. The dashed line indicates that microbial identification can be performed directly using a database of nucleic acid sequences corresponding to microbes associated with a particular metabolic activity, to confirm the presence of the microbial metabolic activity without fully identifying the microbial species present in the sample.

FIG. 3 is a flow diagram depicting how the methods described herein can be used to modulate environmental microbiomes for the purpose of environmental remediation and management of biological oxygen demand (BOD) from factory effluents.

### DETAILED DESCRIPTION

The following description sets forth exemplary methods, conditions, and the like and are not intended as limiting the scope of the present disclosure. Instead, it is provided as a description of exemplary embodiments.

### I. Overview

Disclosed herein are methods and systems for determining microbial metabolic activities in environments and modulating the microbiomes in the environments to alter the microbial metabolic activities present therein. These methods and systems use metagenomic microbiome data from any environmental sample to identify the microbial components within the sample, by using data from high throughput sequencing of nucleic acids. The system can also use microbiome sequence data from smaller scale surveys such as with microarrays or other lower throughput sequencing or PCR. In all cases, as described further below, the invention is based on using metagenomic microbiome data to identify microbial metabolic signatures as indicators the metabolic activities and pathways taking place in the environmental microbiome. The microbial signatures can represent a subset of the metagenomics data obtained for a particular sample. The microbial metabolic signatures can be determined in a targeted manner by analyzing the data to identify a particular set of microbes associated with specific microbial metabolic activities. Alternatively, the sequence data can be analyzed in an unbiased manner to identify microbial metabolic signatures corresponding to detected deviations in the microbiome composition that can then be traced to fluctuations in microbial metabolic activities. This can be applied to environmental safety for the purposes of bioremediation and improved treatment of waste streams to decrease environmental impact. One exemplary use case for these methods is towards aflatoxins that can be reduced or completely degraded to non-toxic metabolites by using the microbiome data to either manipulate conditions to facilitate naturally available bacteria to degrade them or to add specific remediating bacteria that have enhanced capability to degrade aflatoxins. Another exemplary use case is bacteria that metabolize toxic hydrocarbons to non-toxic compounds, where microbiome analyses would facilitate identification of species that can be used to manipulate and control the rate of degradation of hydrocarbons.

The methods and systems described herein solve an important problem in understanding the composition of environmental microbiomes, as well as the complex metabolic activities and pathways occurring therein, to drive their application to practical issues in bioremediation. Most current nucleic acid-based methods for determining specific microbial metabolic activities in an environmental microbiome rely on targeted detection of enzymes known to be involved in particular microbial metabolic pathways. These methods may fail to detect a microbial metabolic activity if the enzymes involved in the metabolic pathway are unknown, or if they are present in an abundance insufficient for detection. The present disclosure is based at least in part on the discovery that microbial metabolic potential and activities may be determined by detecting shifting patterns of microbes within the microbial community, independently of targeted analysis of known enzymes. Thus, the methods and systems described herein can determine a microbial metabolic activity by identifying microbial metabolic signatures that do not comprise nucleic acid sequences encoding known enzymes involved in in the microbial metabolic activity.

Although the following description uses terms first, second, etc., to describe various elements, these elements should not be limited by the terms. These terms are only used to distinguish one element from another.

The terminology used in the description of the various embodiments described herein is for the purpose of describing particular embodiments only and is not intended to be limiting. As used in the description of the various described embodiments and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, rational numbers, steps, operations, elements, and/or components, but do not preclude the presence or addition of one or more other features, integers, rational numbers, steps, operations, elements, components, and/or groups thereof.

The term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

The terms “microbiome” or “microbiota” may be used with meanings and/or intention in the art, but as used in this specification, these terms may be construed to interchangeably encompass any meaning and/or intention used by those in the art, unless otherwise specified.

Metagenomics generally relates to the study of genetic material that is obtained from an environment and allows for analysis of a sample without the need to isolate the genetic material from individual species present in the sample. Metagenomics allows environmental samples to be analyzed in an unbiased, high throughput, and comprehensive manner.

A “microbiome” or “microbiota” generally relates to a community of microbes present in an environment. Shifts in the microbiome/microbiota composition of a sample from an environment can reflect changes in the levels of various microbial metabolic activities in the environment. These changes may result from abiotic perturbations in the environment. For example, pollution of an environment with a toxin can result in an increase in microbial metabolic activities for degradation of that toxin. Analysis of the microbiome/microbiota in a particular environmental sample can be employed to indirectly determine what microbial metabolic activities are active in the environment.

The methods described herein can include identifying one or more microbial metabolic signatures associated with one or more microbial metabolic activities within a plurality of nucleic acid sequences in the one or more environmental samples. As used herein, “microbial metabolic signature” generally refers to a collection of microbial sequences obtained from an environmental sample that indicates the presence and/or the activity (e.g., the rate or the level) of a microbial metabolic activity in the environment sampled. As used herein, a “microbial metabolic activity” generally refers to a metabolic activity carried out by one or more microbes present in an environment. For example, a microbial metabolic activity may be an activity to degrade a toxic or otherwise unwanted compound, an activity to catabolize a specific compound, e.g., a specific sugar, lipid, or protein, or an activity to anabolically biosynthesize a specific compound, e.g., express a specific protein or synthesize a specific small molecule. Importantly, a microbial metabolic signature associated with a microbial metabolic activity may comprise sequences other than sequences encoding enzymes known to be associated with the microbial metabolic activity. In some cases, a microbial metabolic signature associated with a microbial metabolic activity does not comprise any nucleic acid sequences encoding enzymes known to be associated with the microbial metabolic activity. A microbial metabolic signature may correspond to one or more microbes associated with one or more metabolic activities. In some instances, the one or more microbial metabolic signatures correspond to the relative abundance of the one or more microbes associated with the one or more microbial metabolic activities.

The methods described herein may be used to modulate one or more microbial metabolic activities of one or more microbes in an environment. As used herein, the term “modulate one or more microbial metabolic activities” generally refers to altering the presence and/or the activity (e.g., the rate or the level) of a microbial metabolic activity. The presence and/or activity of a microbial metabolic activity may be modulated in an environment by modulating the microbiome in the environment. As used herein, the term “modulating a microbiome in an environment” generally refers to altering the relative abundance of the microbes present in the environment. For example, modulating a microbial metabolic activity in an environment can comprise adding one or more microbes associated with the microbial metabolic activity to the environment in order to increase the relative abundance of microbes performing the metabolic activity in the environment. Modulating a microbial metabolic activity in an environment may also comprise the addition of compounds to the environment to modulate the growth of microbes associated with the metabolic activity. For example, specific substrates may be added to the environment (e.g., sugars) to promote the growth of microbes associated with a particular metabolic activity. Alternatively, antimicrobial compounds may be added to the environment to selectively reduce or inhibit the growth of microbes associated with a particular metabolic activity.

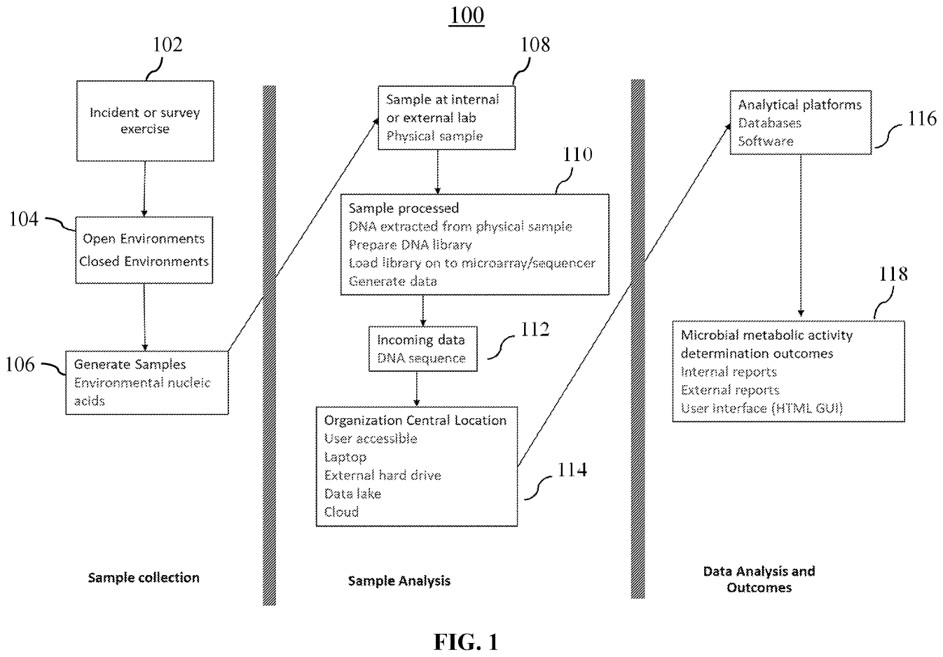

FIGS. 1-3 provide exemplary embodiments of methods for identifying one or more microbial metabolic activities in an environment, wherein the methods comprise obtaining one or more samples from one or more locations in the environment associated with one or more microbial metabolic activities of one or more microbes, sequencing a plurality of nucleic acid sequences within the one or more samples, and identifying one or more microbial metabolic signatures within the plurality of nucleic acid sequences.

FIG. 1 depicts a flowchart of an exemplary method 100 for determining a microbial metabolic activity in an environment. At 102, the process may be initiated by an organization in response to an incident or survey exercise. The incident or survey exercise may be implemented as part of a regular environmental monitoring process or may be implemented to monitor for possible contamination events in response to a change in environmental practice (e.g., a change in a waste stream treatment operation). At 104, the incident or survey exercise prompts the implementation of the method in the environment, for example, in an open environment such as an aquifer or a terrestrial soil, and/or in a closed environment such as in a factory effluent treatment plant. At 106, one or more samples are obtained from one or more locations with the environment or environments identified for testing.

Once obtained, the one or more samples are processed at a designated location, such as an internal or external laboratory (108). Once the physical sample is received by the internal or external laboratory, the sample is processed at step 110. During sample processing, nucleic acids (e.g., DNA) are extracted from the physical sample, a sequencing library (e.g., a DNA library) is prepared, the library is analyzed (e.g., by loading the library onto a microarray or sequencer), and data is generated. Any known method for nucleic acid extraction and library preparation known in the art may be used. For instance, without limitation, nucleic acid extraction may be performed on freshly collected or frozen samples and using any available extraction technique such as phenol: chloroform: isoamyl alcohol extraction or by using any appropriate commercially available kit. The sequencing library may be analyzed using any available technique that provides nucleic acid sequence data, such as, without limitation, next generation sequencing, qPCR, mass spectrometry, chromatography, microarray, in situ sequencing, probe hybridization, and any combination thereof. The method of sequencing library preparation will depend on the analysis technique to be used and may be performed according to the sequencing platform manufacturer's instructions. The generated data is transferred at 112 as incoming data (e.g., DNA sequence data) to a central location in the organization (114). The central location may be user accessible, such as a laptop, an external hard drive, a data lake or a cloud, or any other local or centralized system in the organization, or a data storage location available as a service to the organization. The transferred data may be provided from an internal laboratory or an external source and may be stored in the central location until further downstream analysis.

At 116, the data from the central location is accessed by analytical platforms. The analytical platform may comprise one or more databases or software that enables analysis of the nucleic acid sequence data. Analysis of the nucleic acid sequences may comprise identifying one or more microbial metabolic signatures within the plurality of nucleic acid sequences and comparing the identified microbial metabolic signatures against one or more databases of microbial metabolic signatures that correspond to particular microbial metabolic activities. Analysis of the nucleic acid sequences can further include, without limitation, comparing sequences against one or more additional databases; filtering sequence reads by size, quality, or origin; de-multiplexing a sample; sequence mapping; read quantification; or any combination thereof. Any suitable analytical platform, such as a platform comprising publicly available software or database, or in-house software or databases may be used.

Analysis of the data using the analytical platforms results in one or more microbial metabolic activity determination outcomes (118). The one or more microbial metabolic activity determination outcomes may be included in internal or external reports, which may be reported as a physical report, or displayed in a user interface. For instance, a user interface may display the one or more microbial metabolic activity determination outcomes and allow a user to navigate and refine the outcomes. Additionally, the user interface may allow a user to compare one or more microbial metabolic activity determination outcomes corresponding to different samples from different locations in the environment or samples collected at different timepoints.

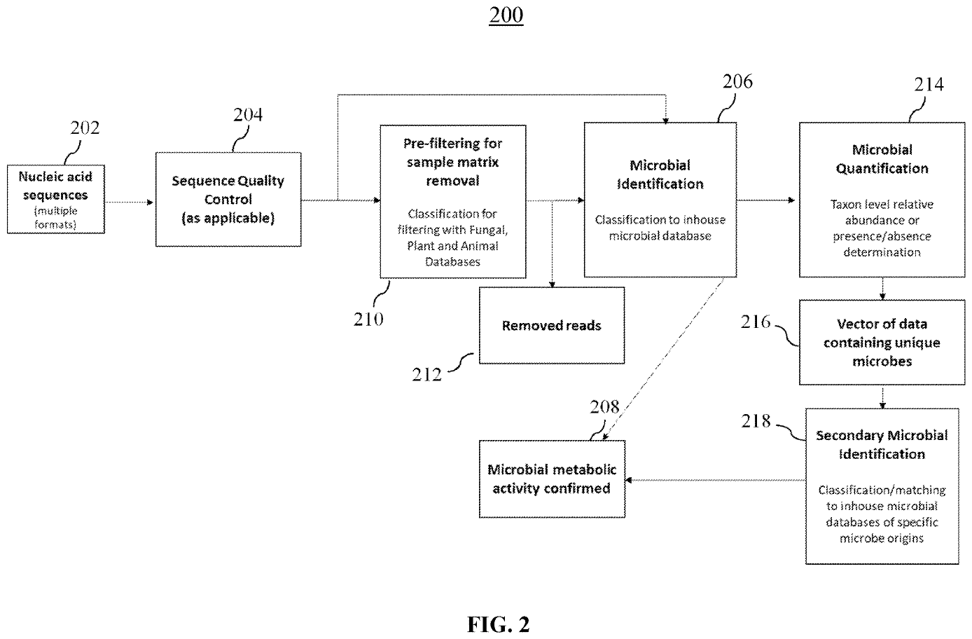

FIG. 2 depicts an exemplary analysis process for identifying one or more microbial metabolic signatures within a plurality of nucleic acid sequences from an environmental sample (method 200). Nucleic acid sequences are received at 202. The nucleic acid sequences may correspond to DNA, RNA, or both DNA and RNA sequences. The nucleic acid sequences may be provided in any suitable format. At 204, a sequence quality control may be implemented as applicable. The sequence quality control may include, without limitation, trimming, length filtering, sequencing adapter removal, sequence binning, or any combination thereof. The nucleic acid sequences are then analyzed for microbial identification (206), in which one or more microbial metabolic signatures are identified.

Microbial identification may include classification to a microbial database (e.g., an in-house microbial database). The microbial databases may be specific to a particular category of microbes, such as a viral database or a fungal database, or may correspond to a wide range of microbes. The microbial databases may also correspond to microbes having particular metabolic activities. Any suitable database corresponding to microbial sequences may be used for microbial identification. The databases may correspond to nucleic acid sequences consisting of combinations of nucleotides (e.g., A, T, G, or C), or they may correspond to amino acid sequences corresponding to one or more protein or protein isoforms encoded by microbial nucleic acids, and which may include any naturally and non-naturally occurring amino acid residues known in the art. After microbial identification, the microbial metabolic activity (208) may be determined based on the one or more microbial metabolic signatures. Prior to microbial identification, a pre-filtering step may be performed for removal of sequences corresponding to the non-microbial sequences, e.g., sequences from the sample matrix or bulk plant material present in aquatic or soil samples (210). The pre-filtering step can include classification of sequences using fungal, plant, or animal databases. In some instances, pre-filtering can identify sequence reads that do not correspond to any microbe present in the sample (e.g., unmapped sequences). At 212, the pre-filtered sequence reads may then be removed from the sequence data.

Microbial quantification (214) may also be performed. The microbial quantification may be determined as the taxon level relative abundance of one or more microbes, or as a presence or absence determination. Microbial quantification may be based on the number of reads corresponding to a particular microbe. For example, a higher read count for sequences corresponding to a particular microbe would indicate higher levels of that microbe in the environment at the location sampled. The quantification may be based on an internal or external control sample. In some instances, microbial quantification may include setting a threshold. For example, the presence or absence of a microbe may be determined based on whether the read count corresponding to that microbe is present in an amount meeting or exceeding a pre-determined threshold. Alternatively, the presence or absence of a microbe in a sample may be determined based on whether sequence reads from that sample surpass a pre-determined threshold, for example, at least 90%, at least 95%, or a 100% sequence identity match to at least one sequence in a reference database. Following microbial quantification, a vector data containing unique microbes is generated (216). The vector data is used for secondary microbial identification at 218. Secondary microbial identification may include, for example, classification or matching of the microbial metabolic signatures to one or more microbial databases. For example, in-house microbial databases corresponding to specific microbial metabolic activities may be used for secondary microbial identification.

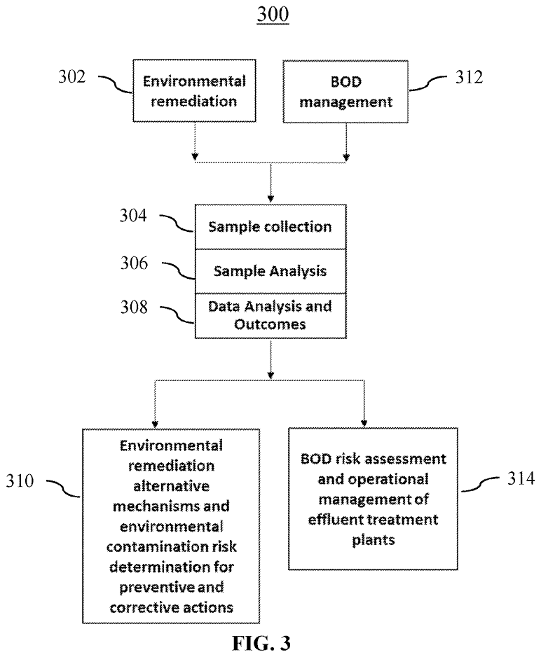

FIG. 3 shows examples of how the methods described herein can be applied to determine and/or modulate microbial metabolic activities in environments for the purpose of environmental remediation and management of biological oxygen demand (BOD) in aquatic environments. The methods described herein may be used for determining and/or modulating microbial metabolic activities in a wide range of environments, including, but not limited to, a soil environment, an aquatic environment, or a factory effluent. Likewise, the methods described herein can be used for determining and modulating a wide range of microbial metabolic activities including, but not limited to, microbial metabolic activities for removing a toxin from an environment, or microbial metabolic activities for lowering BOD in an effluent. At 302, the method is initiated in an environment that has been identified as being in need of remediation, e.g., remediation of a toxic pollutant. Samples are collected from one or more locations in the polluted environment (304), and then the samples are analyzed for microbial metabolic signatures associated with microbial metabolic activities to remove the toxin from the environment (306). The analysis of the microbial metabolic signatures leads to determination of one or more microbial metabolic activities for removing the toxin in the environment (308). As a result, at 310, determination of the one or more microbial metabolic activities for removing the toxin in the environment leads to preventative and corrective actions to remove the toxin from the environment, e.g., by adding microbial species associated with the microbial metabolic activity for removing the toxin to the environment, or by adding substrates to the environment to promote the growth of microbial species associated with the microbial metabolic activity for removing the toxin. At 312, the method is initiated for management of BOD, e.g., in a factory effluent treatment plant for management of BOD in aquatic environments into which the treated effluent is released. Samples are collected from one or more locations in the effluent treatment plant, including from the effluent itself, and optionally from the aquatic environment into which the treated effluent is released (304). The samples are then analyzed for microbial metabolic signatures associated with microbial metabolic activities for lowering BOD (306), and analysis of the microbial metabolic signatures leads to determination of one or more microbial metabolic activities for lowering BOD in the samples (308). As a result, at 314, decisions are made that impact the operations of the effluent treatment plant to increase the microbial metabolic activity for lowering BOD in the effluent, for example, by adding substrates or enzymes to the effluent to promote the growth of microbial species associated with the microbial metabolic activity for lowering, or to reduce the population of microbes not associated with lowering BOD.

Using the methods described above, one or more (e.g., one, two, three, four, five, ten, or more) different microbial metabolic activities may be determined for a particular environment. In some embodiments, the methods may also be performed at multiple locations within an environment or multiple environments, e.g., at multiple locations within a polluted soil or aquifer, or at multiple aquifers. The methods described herein may comprising collecting samples from two three, four, five, ten, or more locations within an environment. Additionally, the methods described herein may comprise collecting samples from two, three, four, five, ten, or more environments.

The methods described herein may be used to modulate one or more microbial metabolic activities of one or more microbes in an environment. For example, in some embodiments, the method comprises determining one or more microbial metabolic activities of one or more microbes in an environment using the methods described herein, and modulating one or more of the determined microbial metabolic activities of the one or more microbes by modulating the microbiome in the environment. The method can comprise repeating the step of determining one or more microbial metabolic activities of one or more microbes in the environment at least once (e.g., one, two, three, four, five, ten, or more times). In some instances, the method can comprise repeating the step of modulating one or more of the determined microbial metabolic activities of the one or more microbes in the environment by modulating the microbiome in the environment at least once (e.g., one, two, three, four, five, ten, or more times).

Modulating the microbial metabolic activity in an environment may comprise modulating the microbiome in the environment, e.g., by altering the relative abundance of one or more microbes present in the environment. The microbiome in an environment may be altered by any means known in the art. For example, modulating the microbiome in an environment may comprise adding microbes to the environment, adding enzymes to the environment, adding metabolite compounds to the environment, altering the conditions of the environment, or any combination thereof. In some instances, the methods herein comprise modulating a microbiome by modulating (e.g., increasing or decreasing) the relative abundance of one or more microbes associated with a particular microbial metabolic activity. This may be achieved by, for example, adding microbes associated with the particular microbial metabolic activity to the environment, by adding metabolite compounds to the environment to promote the growth of microbes associated with the particular metabolic activity, or by adding compounds to inhibit the growth of microbes not associated with the particular metabolic activity. In some embodiments, the method comprises determining a microbial metabolic activity in an environment using a method described herein, identifying one or more microbes in the environment associated with the microbial metabolic activity, isolating and culturing one or more of the identified microbes, and adding the cultured microbes to the environment to increase the relative abundance of the microbes associated with the microbial metabolic activity. In further embodiments, the method comprises determining a microbial metabolic activity in an environment using a method described herein, identifying one or more microbes in the environment associated with the microbial metabolic activity, and adding a metabolite to the environment to promote the growth (either generally or selectively) of the one or more identified microbes associated with the microbial metabolic activity.

### Methods for Removing Toxins From an Environment

The methods herein may be used for removing one or more toxins from an environment by determining one or more microbial metabolic activities for removing the toxin in the environment and modulating the microbiome in the environment to increase one or more of the determined microbial metabolic activities for removing the toxin in the environment. As used herein, the term “toxin” may be construed to encompass any meaning and/or intention used by those in the art, unless otherwise specified. Toxins can include any compound generally understood by those in the art to be harmful to humans, animals, or plants. Toxins can include byproducts from industrial, agricultural, recreational, or waste-treatment processes that are intentionally or unintentionally released into the environment, and can also include toxic compounds that naturally present in the environment, for example, those produced by animal or plant pathogens or by algal blooms. The method may be used to remove toxins from a range of types of environments, including, but not limited to, soil environments and aquatic environments. The method may also be used in such environments where more than one type of toxic component is present, for example, where both aflatoxins and asphaltenes are present. This may be coupled with monitoring the toxin content as a practical application of the aforesaid methods to determine the need for application and/or testing of the environment for its microbiome content.

The methods described herein may be used for removing one or more aflatoxins from an environment. Aflatoxins are a category of toxins that are produced by certain fungi, particularly those of the genus Aspergillus, which grow in soil environments and various crops and staple foods for humans and animals including cereals (e.g., sweetcorn, wheat, and rice), nuts and seeds (e.g., tree nuts, sunflower seeds, sesame seeds, etc.), and hay. Many aflatoxins are carcinogenic and/or mutagenic and have been shown to have toxic effects in humans and animals, and particularly in children, where exposure can lead to liver damage, stunted growth, and delayed development. Aflatoxins include, but are not limited to, aflatoxin B₁ and B₂ (AFB; produced by Aspergillus flavus and A. parasiticus), aflatoxin G₁ and G₂ (AFG; produced by some Group II A. flavus and A. parasiticus), aflatoxin M₁ (AFM₁; a metabolite of aflatoxin B1), aflatoxin M₂ (metabolite of aflatoxin B₂), aflatoxicol (AFL), and Aflatoxin Q₁ (AFQ₁; metabolite of AFB₁).

The methods described herein may also be used for removing one or more toxic hydrocarbons from an environment. Toxic hydrocarbons may be released into the environment as a result of industrial processes including, but not limited to, crude oil extraction and refining operations, manufacturing operations involving the use of petroleum, fuel distribution operations, transportation operations, and agricultural operations. Sources of toxic hydrocarbons can include, without limitation, crude oil, petroleum and its refinement products (e.g., asphaltenes, lube oil waxes, and naphtha), fuels (e.g., gasoline, diesel, jet fuel, kerosene, and propane), and pesticides. Toxic hydrocarbons can include, without limitation: alkanes, both saturated and branched; alkenes, both saturated and branched; naphthalenes, both homo-and heterocyclic; aromatic hydrocarbons, including naphthene aromatics; functionalized hydrocarbons including ethers, carboxylic acids, esters, amides, and the like; asphaltenes; and resins.

The method may comprise determining one or more microbial metabolic activities for removing the one or more toxins in the environment. As used herein, the term “microbial metabolic activity for removing a toxin” generally refers to any microbial metabolic activity that acts to reduce or eliminate animal or plant health hazards associated with the toxin in the environment. Microbial metabolic activities for removing a toxin from an environment can include, but are not limited to, microbial metabolic activities for degrading the toxin, microbial metabolic activities for sequestering the toxin, microbial metabolic activities for neutralizing the toxin, microbial metabolic activities for metabolizing the toxin to produce a non-toxic compound, microbial metabolic activities that reduce the bioavailability of the toxin, or any combination thereof. In some instances, the method comprises determining one or more microbial metabolic activities of one or more microbes for removing the toxin in the environment, e.g., by identifying one or more microbes associated with the determined microbial metabolic activity. In some embodiments, determining one or more microbial activities in the environment comprises obtaining one or more samples from one or more locations in the environment associated with one or more microbial metabolic activities of one or more microbes for removing the toxin, sequencing a plurality of nucleic acid sequences within the one or more samples, identifying one or more microbial metabolic signatures within the plurality of nucleic acid sequences, comparing the one or more identified microbial metabolic signatures against one or more databases of microbial metabolic signatures that correspond to particular microbial metabolic activities of microbes for removing the toxin, and determining one or more microbial metabolic activities of one or more microbes for removing the toxin that correspond to one or more of the identified microbial metabolic signatures based on the comparison. In some instances, the method comprises comprising repeating the step of determining one or more microbial metabolic activities of one or more microbes for removing the toxin in the environment at least once (e.g., one, two, three, four, five, or more times). In some cases, the method may comprise obtaining samples from two or more (e.g., two three, four, five, ten, or more) locations within the environment.

The identified microbial metabolic signature associated with a microbial metabolic activity for removing the one or more toxins may comprise sequences other than sequences encoding enzymes known to be associated with the microbial metabolic activity for removing the toxin. In some cases, a microbial metabolic signature associated with a microbial metabolic activity does not comprise any nucleic acid sequences encoding enzymes known to be associated with the microbial metabolic activity for removing the toxin. For example, in some instances, a microbial metabolic signature may not comprise any nucleic acid sequences encoding enzymes known to be involved in degradation of the toxin, including, but not limited to enzymes known to have the toxin as a substrate, enzymes known to catabolize the toxin or a breakdown product of the toxin, enzymes identified as being part of a pathway for catabolizing or sequestering the toxin, enzymes known to convert the toxin into a less toxic or non-toxic compound, and the like. A microbial metabolic signature may correspond to one or more microbes associated with one or more metabolic activities for removing the toxin. For example, a microbial metabolic signature may correspond to two, three, four, five, ten, or more microbes associated with one or more metabolic activities for removing the toxin. In some instances, the one or more microbial metabolic signatures correspond to the relative abundance of the one or more microbes associated with the one or more microbial metabolic activities for removing the toxin.

The method for removing one or more toxins from an environment may be performed on an environment that has been previously identified as having an unacceptable amount of a toxin and therefore in need of remediation (i.e., removal of the toxin).

Alternatively, the method may comprise identifying the presence of the toxin in the environment, and/or quantifying the amount (e.g., the concentration) of the toxin in the environment, to identify an environment as being in need of remediation (i.e., removal of the toxin). The method may also be used preventatively or anticipatorily in environments where toxic catastrophe or calamities are predicted to happen in short or long term. In some instances, the method comprises repeating the step of identifying the presence of the toxin in the environment and/or quantifying the amount of the toxin in the environment at least once (e.g., one, two, three, four, five, or more times). Methods for identifying the presence and quantifying the amount of toxins in environments are known in the art and may be chosen by one of skill in the art based on the nature of the toxin to be identified. Suitable methods for identifying the presence of the one or more toxins in an environment may include, for example, atomic absorption spectroscopy, colorimetric analysis, elemental analysis, enzymatic assays, fluorescence spectroscopy, gas chromatography-mass spectrometry (GC-MS), high-performance liquid chromatography (HPLC), infrared spectroscopy (IR), light scattering, liquid chromatography-mass spectrometry (LC-MS), liquid chromatography tandem mass spec methods (LC/MS/MS), nuclear magnetic resonance (NMR) spectroscopy, thin layer chromatography (TLC), and ultraviolet-visible (UV/Vis) spectroscopy.

The method for removing one or more toxins from an environment may include modulating one or more microbial metabolic activities of one or more microbes for removing a toxin in an environment. For example, in some embodiments, the method comprises determining one or more microbial metabolic activities of one or more microbes for removing the toxin in an environment using the methods described herein, and removing the toxin by modulating one or more of the determined microbial metabolic activities of the one or more microbes for removing the toxin by modulating the microbiome in the environment. The method can comprise repeating the step of determining one or more microbial metabolic activities of one or more microbes for removing the toxin in the environment at least once (e.g., one, two, three, four, five, ten, or more times). In some instances, the method can comprise repeating the step of removing the toxin from the environment by modulating the microbiome in the environment to increase one or more of the determined microbial metabolic activities for removing the toxin in the environment at least once (e.g., one, two, three, four, five, ten, or more times). The method can also comprise genetic alterations to one or more specific species to increase the rate of metabolism to allow rapid degradation or removal otherwise of the toxic compounds.

Modulating the microbial metabolic activity for removing the one or more toxins in an environment may comprise modulating the microbiome in the environment, e.g., by altering the relative abundance of one or more microbes present in the environment, to increase the rate of removal. In some instances, the method to remove a toxin from an environment comprises modulating a microbiome in the environment by increasing the relative abundance of one or more microbes associated with a particular microbial metabolic activity for removing the toxin. This may be achieved by, for example, adding microbes associated with the particular microbial metabolic activity for removing the toxin to the environment, by adding metabolite compounds to the environment to promote the growth of microbes associated with the particular metabolic activity for removing the toxin (e.g., metabolite compounds that are associated with one or more of the determined microbial metabolic activities for removing the toxin), by adding compounds to inhibit the growth of microbes not associated with the particular metabolic activity for removing the toxin, by adding enzymes to the environment (e.g., enzymes that are associated with one or more of the determined microbial metabolic activities for removing the toxin), or by altering the conditions of the environment. In some embodiments, the method comprises determining a microbial metabolic activity for removing a toxin in an environment using a method described herein, identifying one or more microbes in the environment associated with the microbial metabolic activity for removing the toxin, isolating and culturing one or more of the identified microbes, and adding the cultured microbes to the environment to increase the relative abundance of the microbes associated with the microbial metabolic activity for removing the toxin, thus removing the toxin from the environment. In further embodiments, the method comprises determining a microbial metabolic activity for removing a toxin in an environment using a method described herein, identifying one or more microbes in the environment associated with the microbial metabolic activity for removing the toxin, and adding a metabolite to the environment to promote the growth (either generally or selectively) of the one or more identified microbes associated with the microbial metabolic activity for removing the toxin

### Methods for Lowering Biological Oxygen Demand in Factory Effluents

The methods herein may be used for lowering biological oxygen demand (BOD) in an effluent by determining one or more microbial metabolic activities for lowering BOD in the effluent and modulating the microbiome in the effluent to increase one or more of the determined microbial metabolic activities for lowering BOD in the effluent. BOD is a measure of oxygen availability in aquatic environments and is primarily governed by microbes that survive in such waters aerobically, i.e., by using the dissolved oxygen in the waters. This is relevant for sustaining other aquatic life such as fish species or aquatic plants that use the dissolved oxygen from the water and may not survive at lower oxygen levels.

The lower the BOD, the higher the dissolved oxygen, and the better the chances of survival of other aquatic life. All manufacturing facilities that generate chemical and biological waste need to process the liquid effluent via an effluent treatment plant (ETP) prior to release into aquifers. The incoming fluids to the ETP are typically rich in some specific substrates, as for example, a dairy effluent would be rich in lactose, milk lipids, and milk proteins. Lactose, whey protein, and casein from such effluent are chemically salvaged, but remaining solids may still support microbial growth. The presence of these solids in effluents can result in an increase in BOD in aquifers. A microbial or an enzymatic treatment step to degrade any bio-available solids provides for a much safer effluent produced from the factories, and the methods described herein can be used to guide the treatment of effluent to modulate the microbiome for lowering BOD. This may be coupled with monitoring the BOD as a practical application of the aforesaid methods to determine the need for application and/or testing of the effluent for its microbiome content.

As used herein, the term “effluent” may be construed to encompass any meaning and/or intention used by those in the art, unless otherwise specified. Effluents can include any type of effluent known to one of skill in the art. For example, effluents may include effluents produced by any time of agricultural or industrial operation, including, but not limited to, livestock production operations (e.g., cattle farms, pig farms, and poultry farms), dairy farms and factories, food processing and production facilities (e.g., meat processing facilities and plant-based food processing facilities), leather manufacturing facilities, metal-based equipment manufacturing facilities, and any other kind of chemical and/or biochemical manufacturing facility.

The method may comprise determining one or more microbial metabolic activities for lowering BOD in the effluent. As used herein, the term “microbial metabolic activity for lowering BOD” generally refers to any microbial metabolic activity that acts to maintain a high level of dissolved oxygen in an effluent or aquatic environment and thus reduce or maintain a low BOD in the effluent or environment. Microbial metabolic activities for lowering BOD can include, but are not limited to, microbial metabolic activities for reducing the amount of bio-available compounds and solids in an effluent. For example, microbial metabolic activities for lowering BOD in a food factory effluent (e.g., a dairy factory effluent) can include, but are not limited to, activities for degrading actin, myosin, cellulose, animal lipids, plant proteins, biochemical effluent components, lactose, milk lipids, whey proteins, caseins, bio-available solids, or any combination thereof. In instances where the effluent is a dairy factory effluent, microbial metabolic activities for lowering BOD in a dairy factory effluent can include microbial metabolic activities for degrading or sequestering lactose, whey protein, caseins, milk lipids, bio-available solids, and other milk components without decreasing the concentration of dissolved oxygen in the effluent. In some instances, the method comprises determining one or more microbial metabolic activities of one or more microbes for lowering BOD in the effluent, e.g., by identifying one or more microbes associated with the determined microbial metabolic activity for lowering BOD.

In some embodiments, determining one or more microbial metabolic activities for lowering BOD in the effluent comprises obtaining one or more samples from the effluent associated with one or more microbial metabolic activities of one or more microbes for lowering BOD, sequencing a plurality of nucleic acid sequences within the one or more samples, identifying one or more microbial metabolic signatures within the plurality of nucleic acid sequences, comparing the one or more identified microbial metabolic signatures against one or more databases of microbial metabolic signatures that correspond to particular microbial metabolic activities of microbes for lowering BOD, and determining one or more microbial metabolic activities of one or more microbes for lowering BOD that correspond to one or more of the identified microbial metabolic signatures based on the comparison. In some instances, the method comprises comprising repeating the step of determining one or more microbial metabolic activities of one or more microbes for lowering BOD in the effluent at least once (e.g., one, two, three, four, five, or more times). In some cases, the method may comprise obtaining samples from one or more (e.g., one, two, three, four, five, ten, or more) locations within the effluent treatment plant, the factory that produces the effluent, one or more aquifers into which the treated effluent is released, or any combination thereof. In some embodiments, the identified microbial metabolic signatures correspond to one or more anoxic bacteria associated with one or more microbial metabolic activities for lowering BOD.

The identified microbial metabolic signature associated with a microbial metabolic activity for lowering BOD may comprise sequences other than sequences encoding enzymes known to be associated with the microbial metabolic activity for lowering BOD. In some cases, a microbial metabolic signature associated with a microbial metabolic activity does not comprise any nucleic acid sequences encoding enzymes known to be associated with the microbial metabolic activity for lowering BOD. In some instances, a microbial metabolic signature may not comprise any nucleic acid sequences encoding enzymes known to be involved in degradation of effluent components, including, but not limited to, actin, myosin, cellulose, animal lipids, plant proteins, biochemical effluent components, lactose, milk lipids, whey proteins, caseins, bio-available solids, or any combination thereof. For example, microbial metabolic signatures for lowering BOD in a dairy factory effluent may not comprise any nucleic acid sequences encoding enzymes known to degrade or sequester lactose, whey protein, caseins, milk lipids, bio-available solids, and other milk components without decreasing the concentration of dissolved oxygen in the effluent. A microbial metabolic signature may correspond to one or more microbes associated with one or more metabolic activities for lowering BOD. For example, a microbial metabolic signature may correspond to two, three, four, five, ten, or more microbes associated with one or more metabolic activities for lowering BOD. In some instances, the one or more microbial metabolic signatures correspond to the relative abundance of the one or more microbes associated with the one or more microbial metabolic activities for lowering BOD.

The method for lowering BOD in an effluent may be performed on an effluent that has been previously identified as having an unacceptably high BOD and therefore in need of improved treatment to lower BOD. Alternatively, the method may comprise quantifying the BOD of one or more effluents to identify one or more of the effluents as having an unacceptably high BOD and therefore in need of improved treatment to lower BOD. In some instances, the method comprises repeating the step of quantifying the BOD of one or more effluents at least once (e.g., one, two, three, four, five, or more times).

The method for lowering BOD in an effluent may include modulating one or more microbial metabolic activities of one or more microbes for lowering BOD in the effluent. For example, in some embodiments, the method comprises determining one or more microbial metabolic activities of one or more microbes for lowering BOD in an effluent using the methods described herein, and lowering BOD in the effluent by modulating the microbiome in the effluent to increase one or more of the determined microbial metabolic activities for lowering BOD in the effluent. The method can comprise repeating the step of determining one or more microbial metabolic activities of one or more microbes for lowering BOD in the effluent at least once (e.g., one, two, three, four, five, ten, or more times). In some instances, the method can comprise repeating the step of lowering BOD in the effluent by modulating the microbiome in the effluent to increase one or more of the determined microbial metabolic activities for lowering BOD in the effluent at least once (e.g., one, two, three, four, five, ten, or more times). The method can also comprise genetic alterations to one or more specific species to increase the rate of metabolism to allow rapid degradation or removal otherwise of the unwanted compounds, aiding the reduction of the BOD.

Modulating the microbial metabolic activity for lowering BOD in an effluent may comprise modulating the microbiome in the effluent, e.g., by altering the relative abundance of one or more microbes present in the effluent. In some instances, the method to lower BOD in an effluent comprises modulating a microbiome in the effluent by increasing the relative abundance of one or more microbes associated with a particular microbial metabolic activity for lowering BOD. This may be achieved by, for example, adding microbes associated with the particular microbial metabolic activity for lowering BOD to the effluent, by adding metabolite compounds to the effluent to promote the growth of microbes associated with the particular metabolic activity for lowering BOD (e.g., metabolite compounds that are associated with one or more of the determined microbial metabolic activities for lowering BOD), by adding compounds to inhibit the growth of microbes not associated with the particular metabolic activity for lowering BOD, by adding enzymes to the effluent (e.g., enzymes that are associated with one or more of the determined microbial metabolic activities for lowering BOD), or by altering the conditions of the effluent and/or the effluent treatment plant. In some embodiments, the method comprises determining a microbial metabolic activity for lowering BOD in an effluent using a method described herein, identifying one or more microbes in the effluent associated with the microbial metabolic activity for lowering BOD, isolating and culturing one or more of the identified microbes, and adding the cultured microbes to the effluent to increase the relative abundance of the microbes associated with the microbial metabolic activity for lowering BOD, thus lowering BOD in the effluent. In further embodiments, the method comprises determining a microbial metabolic activity for lowering BOD in an effluent using a method described herein, identifying one or more microbes in the effluent associated with the microbial metabolic activity for lowering BOD, and adding a metabolite to the effluent to promote the growth (either generally or selectively) of the one or more identified microbes associated with the microbial metabolic activity for lowering BOD. In some instances, modulating the microbiome in the effluent comprises adding anoxic bacteria to the effluent, e.g., anoxic bacteria associated with one or more of the determined microbial metabolic activities for lowering BOD.

### II. Nucleic Acid Sequencing

The methods disclosed herein comprise sequencing a plurality of nucleic acid sequences within the one or more environmental samples. The nucleic acid sequences may correspond to any nucleic acid present in a sample. For instance, the nucleic acid sequences may correspond to a plurality of DNA and/or RNA sequences. The plurality of nucleic acid sequences may correspond to nucleic acids from one or more microbes present in an environmental sample, or to other nucleic acids present in the sample, e.g., nucleic acids from non-microbial organisms such as plants present in aquatic or soil samples.

Sequencing a plurality of nucleic acid sequences within the one or more samples can include extracting nucleic acids from the sample. Methods of extracting nucleic acids known in the art may be used. Without being limited, nucleic acids may be extracted using TrizolLS reagent, phenol: chloroform: isoamyl alcohol extraction, or equivalents. Nucleic acid extraction may also be performed using commercially available kits, such as, Ambion RNA isolation kits (e.g., Purelink RNA Mini kit or DynaBeads mRNA direct micro kit), MAgmax FFPE total nucleic acid isolation kit, Pall DNA and RNA Purification kits, Qiagen Allprep, Power Viral, Powersoil, or PowerMag kits, NEBNext Microbiome DNA Enrichment kit, or equivalents. Nucleic acid extraction may be performed using frozen or fresh samples. Nucleic acid extraction may also include a step of cell lysis. Cell lysis may be performed through any methods known to those skilled in the art, including, but not limited to, enzymatic lysis using lytic enzymes such as lysozyme, lysostaphin, mutanolysin, proteinase K, subtilisin, or any combination thereof; physical shearing, such as with glass beads, sonication, ultrasound, or high pressure (e.g., using French press); and any other cell lysis method known to those skilled in the art.

It should be understood that the present teachings contemplate sequencing a plurality of nucleic acid sequences within the one or more samples using all available varieties of techniques, platforms, or technologies, including, but not limited to: capillary electrophoresis, microarrays, ligation-based systems, polymerase-based systems, hybridization-based systems, in situ sequencing, direct or indirect nucleotide identification systems, pyrosequencing, ion- or pH-based detection systems, electronic signature-based systems, etc.

The plurality of nucleic acid sequences within the one or more samples may be sequenced by any method available in the art, such as by nucleic acid sequencing (e.g., next generation sequencing) or microarray analysis. The methods disclosed herein are not dependent upon a particular next generation sequencing technology, and the user needs to make appropriate choices for the intended downstream sequencing platform according to manufacturers' protocols. Exemplary sequencing platforms that may be used to obtain sequence data according to the methods disclosed herein include, but are not limited to, those produced by Illumina®, Oxford Nanopore™, Ion Torrent™, Roche™, Pacific Biosciences™, and Life Technologies™.

Depending on the sequencing technology used with the methods, a sequencing library may be prepared. The sequencing library will be representative of nucleic acids present in a sample and can be used with next generation sequencing platforms. Sequencing library preparation can include nucleic acid fragmentation, sample indexing, adaptor ligation, and library normalization. Sample indexing or barcoding allows multiple samples to be run simultaneously, taking full advantage of the high-throughput nature of current sequencing platforms. Adapter ligation is sequencing platform specific and standard to manufacturers' protocols. The adaptors may contain sequencing platform-specific end sequences and index sequences that allow for de-convolution of sequence data by sample. Barcoding and adapter ligation may be performed by any method known to those in the art, and may be adapted for analysis of the sequencing library with a particular sequencing platform. Library preparation can also include amplification, concentration, or dilution of the sequencing library. Libraries can be prepared at platform-specific concentrations of DNA and typically require amplification, concentration, or dilution to achieve the required concentration. The concentration of nucleic acids in the sequencing library may be determined by quantitative real-time PCR using platform specific manufacturer protocols or fluorescence-based measurement known in the art. In some instances, preparing the sequencing library includes selective enrichment of specific target nucleic acids or regions.

Nucleotide sequences of individual molecules are determined in a platform-specific manner to produce a raw dataset. The raw dataset can be converted to nucleotide sequencing information corresponding to each molecule in a sequencing library. The resulting products are whole “reads,” which may be processed to determine information about the environment. The sequence data may be produced in any format, such as BAM files, which are sequencing platform-independent and ready for bioinformatics analysis. Additional file types may include FASTA and FASTQ file formats, or other manufacturer-specific formats that can be converted to BAM, VCF, FASTQ, or FASTA format.

Once obtained, sequence data containing the plurality of nucleic acid sequences may be transferred in real time from the instrument used to generate sequence data as soon as the sequence data has reached a sufficient size in total base pairs for analysis, or it may be stored in a database until further analysis. Data may be stored on any suitable database or device, including, but not limited to, on a server, on a personal computer, on a smartphone, on a tablet, on a cloud system (e.g., AWS™, Google Cloud™, or MS Azure™), or on a local hard drive (e.g., an external hard drive).

The sequence data containing the plurality of nucleic acid sequences may then be prepared for further analysis. This preparation can include performing sequence quality control, trimming, length filtering, sequencing adapter removal, and/or binning of reads by molecular barcode from the sequencing reads. In particular, the reads that represent the plurality of nucleic acid sequences from the sample can be quality controlled to remove the adapter sequences, clonal reads due to PCR amplification, and platform-specific sequence errors and filtered to achieve an acceptable error rate. Sequencing reads in the sequencing data may be deconstructed into, for example, k-mers of a particular size. Sequence assembly, mapping, or pairwise comparison of the sequencing reads in the sequence data may also be performed. In some cases, nucleic acid sequences corresponding to the sample matrix or other non-microbial biomass can be filtered or removed from the sequence data prior to further analysis. For example, if the sample is a soil sample, nucleic acid sequences corresponding to the plant biomass present in the soil sample can be removed from the sequence data prior to further analysis.

### III. Microbial Metabolic Signatures

The methods described herein can include identifying one or more microbial metabolic signatures associated with one or more microbial metabolic activities within the plurality of nucleic acid sequences in the one or more samples. The one or more microbial metabolic signatures may correspond to one or more microbes present in the sample and may indicate the presence or the activity of one or more microbial metabolic activities present in the sample. Importantly, a microbial metabolic signature associated with a microbial metabolic activity may comprise sequences other than sequences encoding enzymes known to be associated with the microbial metabolic activity. In some cases, a microbial metabolic signature associated with a microbial metabolic activity does not comprise any nucleic acid sequences encoding enzymes known to be associated with the microbial metabolic activity. A microbial metabolic signature may correspond to one or more microbes associated with one or more metabolic activities. For example, a microbial metabolic signature may correspond to two, three, four, five, ten, or more microbes associated with one or more metabolic activities. In some instances, the one or more microbial metabolic signatures correspond to the relative abundance of the one or more microbes associated with the one or more microbial metabolic activities. The microbial metabolic signatures may correspond to a particular taxonomic level, such as a kingdom, a phylum, a class, a genus, a species, a serotype, or a strain. The microbes present in the sample (including any unwanted microbes) may be microbial eukaryotes, bacteria, archaea, fungi, or viruses.

Identifying the one or more microbial metabolic signatures can include comparing sequence data to one or more databases. The databases can contain sequences (e.g., nucleic acid sequences, or amino acid sequences) from a particular group of microbes. For instance, the databases may correspond to nucleic acid sequences from microbes that are associated with one or more particular microbial metabolic activities. These databases may correspond to a specific microbial taxon, a specific genus, or a collection of microbial species. Any publicly available database that is suitable for microbe identification may be used. Alternatively, an in-house database may be generated and used to identify a microbial metabolic signature.

Identifying one or more microbial metabolic signatures can include determining the relative level of nucleic acids in the sequence data corresponding to one or more particular microbes in the sample. The relative level of the nucleic acids in the microbial metabolic signature can be indicative of the relative level of the particular microbes and may be used to determine the relative abundance of particular microbes. A threshold may be set, and any indicator microbial signature corresponding to a relative level of one or more microbes above the predetermined threshold would indicate the presence and/or abundance of an unwanted microbe. The threshold may be set in terms of, for example, a Ct value, a nucleic acid copy number, a concentration (e.g., in mg/mL or mg/L units), etc.

Microbial metabolic signatures may correspond to one or more of a bacteria, virus, archaea, or eukaryotic microorganisms. Exemplary microbes that can be present in an environmental sample include, but are not limited to, microbes belonging to a genus taxonomy selected from the group consisting of Parageobacillus, Blautia, Aliivibrio, Porphyrobacter, Shigella, Aneurinibacillus, Anaerostipes, Photobacterium, Erythrobacter, Rathayibacter, Butyrivibrio, Tyzzerella, Grimontia, Dechloromonas, Leifsonia, Coprothermobacter, Intestinimonas, Pseudoalteromonas, Pseudarthrobacter, Arthrobacter, Megasphaera, Ethanoligenens, Alteromonas, Isoptericola, Micrococcus, Eubacterium, Colwellia, Cellulomonas, Thermus, Oscillibacter, Yersinia, Nocardia, Meiothermus, Weissella, Edwardsiella, Gordonia, Rahnella, Murdochiella, Oceanimonas, Propionibacterium, Azotobacter, Eggerthella, Marinomonas, Tessaracoccus, Caulobacter, Adlercreutzia, Halomonas, Pimelobacter, Fibrobacter, Gordonibacter, Methylophaga, Actinoplanes, Fervidobacterium, Obesumbacterium, Brucella, Listeria, Methanobrevibacter, Plesiomonas, Caldanaerobacter, Deinococcus, Methanosarcina, Gallibacterium, Synechococcus, Spirosoma, Thioploca, Calothrix, Helicobacter, Thermotoga, Janthinobacterium, Nonlabens, Barnesiella, Fusobacterium, Ornithobacterium, Ilyobacter, Akkermansia, Thermodesulfobacterium, Cloacibacillus, Theileria, Gyrovirus, T7virus, T4virus, Alpharetrovirus, Sp18virus, Acidaminococcus, Altererythrobacter, Comamonas, Arcobacter, Aeromicrobium, Pediococcus, Proteus, Alistipes, Azospira, Geobacillus, Geoalkalibacter, Agrobacterium, Vibrio, Christensenella, Bosea, Kurthia, Hafnia, Alcaligenes, Clostridioides, Novosphingobium, Oblitimonas, Morganella, Amycolatopsis, Odoribacter, Pseudoxanthomonas, Negativicoccus, Aureimonas, Olsenella, Psychrobacter, Paenibacillus, Brachybacterium, Parabacteroides, Shewanella, Providencia, Brevibacterium, Roseburia, Candida, Ruminococcus, Caulimovirus, Selenomonas, Clavibacter, Treponema, Curtobacterium, Turicibacter, Erwinia, Frondihabitans, Hymenobacter, Kineococcus, Kluyveromyces, Massilia, Methylobacterium, Microbacterium, Nocardioides, Ochrobactrum, Pseudonocardia, Rhizobium, Saccharopolyspora, Sanguibacter, Shinella, Sphingobacterium, Sugiyamaella, Chryseobacterium, Aeromonas, Achromobacter, Blastomonas, Pantoea, Delftia, Anoxybacillus, Bordetella, Mycobacterium, Bacteroides, Brevundimonas, Rhodococcus, Bifidobacterium, Kosakonia, Streptomyces, Desulfovibrio, Sphingobium, Thermothelomyces, Flavonifractor, Sphingomonas, Thielavia, Lachnoclostridium, Sphingopyxis, Macrococcus, Cupriavidus, Moraxella, Prevotella, Ruminiclostridium, Bradyrhizobium, Campylobacter, Clostridium, Stenotrophomonas, Burkholderia, Cutibacterium, Xanthomonas, Serratia, Escherichia, Staphylococcus, Streptococcus, Variovorax, Acidovorax, Acinetobacter, Bacillus, Citrobacter, Corynebacterium, Enterobacter, Enterococcus, Klebsiella, Lactobacillus, Lactococcus, Pseudomonas, Raoultella, and Salmonella.A microbial metabolic signature may correspond to any microbe of the foregoing taxonomic designations, either singly or in any combination.

### IV. Systems

In one aspect, the disclosure provides systems for performing any of the methods described herein.

In some embodiments, the system can be configured to determine one or more microbial metabolic activities in an environment, e.g., a microbial metabolic activity for removing a toxin or a microbial metabolic activity for lowering biological oxygen demand (BOD) in an effluent. For example, the system may include one or more processors and a memory comprising instructions executable by the one or more processors. When executed by the one or more processors, the instructions may cause the system to identify one or more microbial metabolic signatures within a plurality of nucleic acid sequences obtained from sequencing a plurality of nucleic acid sequences within one or more samples from one or more locations in the environment associated with one or more microbial metabolic activities of one or more microbes, compare the one or more identified microbial metabolic signatures against one or more databases of microbial metabolic signatures that correspond to particular microbial metabolic activities of microbes, and determine one or more microbial metabolic activities of one or more microbes that correspond to one or more of the identified microbial metabolic signatures based on the comparison.

### V. Methods in Computer-readable Storage Devices

Any of the methods described herein can be implemented by computer-executable instructions or code stored in one or more computer-readable medium (e.g., a memory, a magnetic storage, an optical storage, or the like). Such instructions can cause one or more processors to implement the method.

### EXAMPLES

### Example 1: Metagenomic Data Filtering for Environmental Remediation Facilitating Bacteria

In this Example, the methods described herein are used for identifying microbial metabolic signatures that are associated with specific metabolic activities to allow tracking and modulation of microbes capable degrading toxic materials. First, the presence of a toxic material in an environment is identified using any suitable method known to one of skill in the art, identifying the environment as polluted. Then, one or more environmental samples are obtained from one or more locations in the polluted environment. Nucleic acids are extracted from the samples and sequenced using methods known in the art and described herein. The resulting sequencing data are analyzed for microbial metabolic signatures, which are identified and compared to a database of microbial metabolic signatures associated with particular microbial metabolic activities for removing the toxin from the environment. The analysis results in determination of the presence or absence of one or more microbial metabolic activities for removing the toxin from the environment. This determination in turn informs decisions for increasing the microbial metabolic activity for removing the toxin from the environment, for example, by adding microbial species associated with the microbial metabolic activity for removing the toxin to the environment, or by adding substrates to the environment to promote the growth of microbial species associated with the microbial metabolic activity for removing the toxin.

### Example 2: Metagenomic Data Filtering for Lowering Biological Oxygen Demand in Factory Effluents Using Targeted Species Addition

In this Example, the methods described herein are used for identifying microbial metabolic signatures that are associated with specific metabolic activities to allow modulation of microbiomes in factory effluents to lower biological oxygen demand (BOD) in the effluents. First, the BOD of a factory effluent is measured using any suitable method known to one of skill in the art, identifying the effluent as having unacceptably high BOD. Then, one or more samples from are obtained from the effluent and, optionally, from one or more locations in the factory, the effluent treatment plant, and/or the aquifer that is impacted by the effluent. Nucleic acids are extracted from the samples and sequenced using methods known in the art and described herein. The resulting sequencing data are analyzed for microbial metabolic signatures, which are identified and compared to a database of microbial metabolic signatures associated with particular microbial metabolic activities for lowering BOD in aquatic environments. The analysis results in determination of the presence or absence of one or more microbial metabolic activities for lowering BOD. This determination in turn informs decisions for increasing the microbial metabolic activity for lowering BOD, for example, by adding microbial species associated with the microbial metabolic activity to the effluent, or by adding substrates or enzymes to the effluent to promote the growth of microbial species associated with the microbial metabolic activity, or to reduce the population of microbes not associated with lowering BOD. This may include adding enzymes or anoxic bacteria to modify the microbiome of the effluent to reduce BOD.

1. A method for determining one or more microbial metabolic activities of one or more microbes in an environment, the method comprising:
obtaining one or more samples from one or more locations in the environment associated with one or more microbial metabolic activities of one or more microbes;
sequencing a plurality of nucleic acid sequences within the one or more samples;
identifying one or more microbial metabolic signatures within the plurality of nucleic acid sequences;
comparing the one or more identified microbial metabolic signatures against one or more databases of microbial metabolic signatures that correspond to particular microbial metabolic activities of microbes; and
determining one or more microbial metabolic activities of one or more microbes that correspond to one or more of the identified microbial metabolic signatures based on the comparison.

2. A method for modulating one or more microbial metabolic activities of one or more microbes in an environment, the method comprising:
determining one or more microbial metabolic activities of one or more microbes in the environment according to the method of claim 1, and
modulating one or more of the determined microbial metabolic activities of the one or more microbes in the environment by modulating the microbiome in the environment.

3. The method of claim 2, further comprising repeating the step of determining one or more microbial metabolic activities of one or more microbes in the environment at least once.

4. The method of claim 2, further comprising repeating the step of modulating one or more of the determined microbial metabolic activities of the one or more microbes in the environment by modulating the microbiome in the environment at least once.

5. The method of claim 1, wherein the one or more microbial metabolic signatures comprise nucleic acid sequences other than sequences encoding enzymes known to be associated with the one or more microbial metabolic activities.

6. The method of claim 1, wherein the one or more microbial metabolic signatures do not comprise a nucleic acid sequence encoding an enzyme known to be associated with one or more of the microbial metabolic activities.

7. The method of claim 1, wherein the one or more microbial metabolic signatures correspond to one or more microbes associated with the one or more microbial metabolic activities.

8. The method of claim 7, wherein the one or more microbial metabolic signatures correspond to two, three, four, five, ten, or more microbes associated with the one or more microbial metabolic activities.

9. The method of claim 7, wherein the one or more microbial metabolic signatures correspond to the relative abundance of the one or more microbes associated with the one or more microbial metabolic activities.

10. The method of claim 2, wherein modulating the microbiome in the environment comprises adding microbes to the environment, adding enzymes to the environment, adding metabolite compounds to the environment, adding substrates to the environment, altering the conditions of the environment, or any combination thereof to modulate one or more of the determined microbial metabolic activities.

11. The method of claim 10, wherein modulating the microbiome comprises adding microbes, enzymes, substrates, and/or metabolite compounds that are associated with one or more of the determined microbial metabolic activities.

12. The method of claim 1, wherein the one or more microbial metabolic activities comprise a microbial metabolic activity for removing one or more toxins from an environment or for lowering biological oxygen demand (BOD) in an effluent.

13. A method for removing one or more toxins from an environment, the method comprising:
identifying the presence of the one or more toxins in the environment;
determining one or more microbial metabolic activities of one or more microbes for removing the one or more toxins in the environment by:
obtaining one or more samples from one or more locations in the environment associated with one or more microbial metabolic activities of one or more microbes for removing the one or more toxins;
sequencing a plurality of nucleic acid sequences within the one or more samples;
identifying one or more microbial metabolic signatures within the plurality of nucleic acid sequences;
comparing the one or more identified microbial metabolic signatures against one or more databases of microbial metabolic signatures that correspond to particular microbial metabolic activities of microbes for removing the one or more toxins; and
determining one or more microbial metabolic activities of one or more microbes for removing the one or more toxins that correspond to one or more of the identified microbial metabolic signatures based on the comparison, and removing the one or more toxins from the environment by modulating the microbiome in the environment to increase one or more of the determined microbial metabolic activities for removing the one or more toxins in the environment.

14-26. (canceled)

27. A method for lowering biological oxygen demand (BOD) in an effluent, the method comprising:
determining one or more microbial metabolic activities of one or more microbes for lowering BOD in the effluent by:
obtaining one or more samples from the effluent associated with one or more microbial metabolic activities of one or more microbes for lowering BOD;
sequencing a plurality of nucleic acid sequences within the one or more samples;
identifying one or more microbial metabolic signatures within the plurality of nucleic acid sequences;
comparing the one or more identified microbial metabolic signatures against one or more databases of microbial metabolic signatures that correspond to particular microbial metabolic activities of microbes for lowering BOD; and
determining one or more microbial metabolic activities of one or more microbes for lowering BOD that correspond to one or more of the identified microbial metabolic signatures based on the comparison, and lowering BOD in the effluent by modulating the microbiome in the effluent to increase one or more of the determined microbial metabolic activities for lowering BOD in the effluent.

28-37. (canceled)

38. The method of claim 1, wherein sequencing the plurality of nucleic acid sequences within the one or more samples comprises preparing a sequencing library.

39. The method of claim 1, wherein sequencing the plurality of nucleic acid sequences within the one or more samples comprises next generation sequencing or microarray analysis.

40. The method of claim 1, wherein the plurality of nucleic acid sequences comprise DNA sequences, RNA sequences, or a combination thereof.

41. (canceled)

42. The method of claim 1, wherein non-microbial sequences are filtered from the plurality of nucleic acid sequences prior to identifying the one or more microbial metabolic signatures.

43. The method of claim 1, wherein the one or more databases are databases of microbial nucleic acid sequences.

44. A system for carrying out the method of claim 1, comprising:
one or more processors; and
a memory comprising instructions executable by the one or more processors that, when executed by the one or more processors, cause the system to:
identify one or more microbial metabolic signatures within a plurality of nucleic acid sequences obtained from sequencing a plurality of nucleic acid sequences within one or more samples from one or more locations in the environment associated with one or more microbial metabolic activities of one or more microbes;
compare the one or more identified microbial metabolic signatures against one or more databases of microbial metabolic signatures that correspond to particular microbial metabolic activities of microbes; and
determine one or more microbial metabolic activities of one or more microbes that correspond to one or more of the identified microbial metabolic signatures based on the comparison.

45. (canceled)
